(19) United States
(12) Patent Application Publication
(10) Pub. No.: US 2018/0325058 A1
(43) Pub. Date: Nov. 15, 2018

(54) NON-TRANSGENIC HAPLOID INDUCER LINES IN CUCURBITS

(71) Applicant: RIJK ZWAAN ZAADTEELT EN ZAADHANDEL B.V., De Lier (NL)

(72) Inventors: Cornelis Maria Petrus VAN DUN, De Lier (NL); Cecilia Lucia Clara LELIVELT, De Lier (NL); Sara MOVAHEDI, De Lier (NL)

(21) Appl. No.: 15/774,782

(22) PCT Filed: Nov. 8, 2016
(86) PCT No.: PCT/EP2016/076987

§ 371 Date: May 9, 2018

(30) Foreign Application Priority Data
Nov. 9, 2015 (NL) 2015753

(51) Int. Cl. A01H 1/08; A01H 5/08; A01H 6/34

(52) U.S. Cl. CPC A01H 1/08; A01H 6/34; A01H 5/08

(57) ABSTRACT

The present invention relates to a mutant plant of the Cucurbitaceae family comprising a modified CENH3 gene, which mutant plant, when crossed to a wild-type plant having 2n chromosomes produces progeny, at least 0.1% of which have n chromosomes. Preferably, the modified CENH3 gene comprises at least one mutation compared to an otherwise identical naturally occurring CENH3 gene, which at least one mutation gives rise to at least one non-conservative amino acid change in the Histone Fold Domain of the encoded modified CENH3 protein or to the occurrence of a premature stop codon in the encoded modified CENH3 protein. The invention further relates to a method for the production of haploid or doubled haploid plants and to the plants thus obtained.

The present invention relates to a mutant plant of the Cucurbitaceae family that can be used as a non-transgenic haploid inducer line. The invention further relates to parts of the plant, and to progeny of the plant.

In plant breeding, the main goal is to combine as many desirable traits as possible in a single genome, while at the same time eliminating as many undesirable traits as possible. This is a slow process that requires the crossing of many individual lines, evaluating the outcome of such crosses during the course of several growth seasons, and selecting promising offspring for further research. Often a selected line displays a few very good characteristics (such as, for example, larger fruits, drought tolerance, disease resistance, faster germination capacity, etc), but also many suboptimal properties that would not be accepted by the consumer and/or by the plant grower. The interesting characteristics of the selected line then need to be introduced into a commercially acceptable genetic background, without losing any of the commercially important traits, to eventually end up with a pure breeding line, in which all desired traits are genetically fixed. This endeavour typically requires multiple generations of backcrossing, because genetically unlinked traits tend to segregate away from each other, and this is therefore a very slow process. Depending on the average generation time (from seed to seed) of the species the creation of a new plant variety may take between 8 and 20 years. A pure breeding line can e.g. be used as a parent of a hybrid variety. Two inbred lines (whose genomes are highly homozygous) are crossed to each other, and the resulting hybrid seeds are sold. Hybrid lines usually display a combination of the superior characteristics of their parents, and they often outperform both their parents due to the high heterozygosity of their genome (hybrid vigour).

Plant breeding can be accelerated through the use of Doubled Haploid (DH) lines, which have a fully homozygous genome within a single generation. An important advantage of DHs is that they are fertile and can be sexually propagated indefinitely.

DHs can be created from the spores of a plant by means of e.g. androgenesis or gynogenesis protocols, or through the use of haploid inducer systems. The genome of these haploid plants is subsequently doubled, which explains why they are completely homozygous. Genome doubling can either occur spontaneously, or it can be induced through the addition of mitosis-blocking chemicals such as colchicine, oryzalin or trifluralin. This leads to the formation of doubled haploid plants (DH plants, DHs), which are able to produce seeds. In this manner the doubled haploid lines are immortalised. Each DH line represents one specific combination of traits derived from the parents of the starting plant, resulting from the reshuffling of all genetically unlinked traits during meiosis.

DHs can be produced from the spores of a starting plant by first creating haploid plants of the spores by means of androgenesis, such as microspore culture or anther culture, by gynogenesis, or by inducing the loss of maternal or paternal chromosomes from a zygote resulting from a fertilisation event, and then doubling the genome of the haploid plants thus obtained. The skilled person is very familiar with these methods of DH production, and he knows which method works best in his favourite plant species. Genome doubling may occur spontaneously, or it may be induced by the application of chemicals, such as colchicine, oryzalin or trifluralin. These chemicals disrupt spindle formation during mitosis, and are typically used for the blocking of mitosis.

The loss of maternal chromosomes from a zygote resulting from a fertilisation event can be induced by using a haploid inducer line as the female in a cross. Haploid inducer systems have been described in various plant species, for example when the female crossing partner is a plant of a different species than the male crossing partner. In interspecific crosses, loss of the genome of one of the parents has often been observed, such as in the cross between wheat and pearl millet, between barley and Hordeum bulbosum, and between tobacco (Nicotiana tabacum) and Nicotiana africana.

For members of the Cucurbitaceae family, protocols are available for the efficient in vitro production of DHs (see e.g. Galązka & Niemirowicz-Szczytt 2013, Folia Hort. 25: 67-78; U.S. Pat. No. 5,492,827). However, DH protocols are not applicable to all genotypes, and several types of Cucurbits are not amenable to standard in vitro haploid induction techniques. It has not been possible to obtain DHs in vivo, as interspecific crosses leading to the loss of one of the parental genomes have not been described. Producing DHs in vivo has clear logistic advantages over the in vitro approaches: it is less labour-intensive, and it does not require a cell biology laboratory or controlled growth facilities for the sterile cultivation of plant material.

It is therefore an object of the current invention to provide an in vivo haploid inducer system for plants belonging to the Cucurbitaceae family.

In the literature, an in vivo system for obtaining haploid plants through genome elimination has been described for Arabidopsis thaliana. This system is based on the transgenic expression of a recombinantly altered CENH3 (centromeric histone H3) polypeptide in a plant having a corresponding inactivated endogenous CENH3 gene (Maruthachalam Ravi & Simon W. L. Chan; Haploid plants produced by centromere-mediated genome elimination; Nature 464 (2010), 615-619; US-2011/0083202; WO2011/044132). CENH3 is a centromeric histone protein that is part of the kinetochore complex, and it plays an important role in chromosome segregation during mitosis and meiosis. CENH3 consists of a highly variable N-terminal tail domain and a conserved histone fold domain (HFD). Swapping the N-terminal tail domain of Arabidopsis CENH3 with that of another histone and the concurrent fusion to Green Fluorescent Protein (GFP) results in a situation wherein Arabidopsis plants expressing this recombinant fusion protein are partially sterile. When crossed to a wild-type Arabidopsis plant, the chromosomes of the parent expressing this recombinant fusion protein missegregate during embryogenesis, resulting in the elimination of the corresponding parental genome and the production of haploid plants whose chromosomes were solely derived from the wild-type parent. Genome doubling can subsequently be achieved as described above. CENH3 appears to be an essential gene, as null mutants in Arabidopsis display embryonic lethality.

The haploid plants produced by this approach are however considered to be transgenic, receiving a Genetically Modified Organism (GMO) status, according to the current legislation in e.g. Europe, even though they themselves do not contain a transgenic construct. For any line with a GMO status to receive approval for commercial use and animal and/or human consumption, it needs to undergo extensive regulatory procedures, which are tremendously expensive and time-consuming. Moreover, in important parts of the worldwide food market, transgenic food is not allowed for human consumption, and not appreciated by the public.

It is therefore a further object of the current invention to provide an in vivo haploid inducer system for plants belonging to the Cucurbitaceae family, that gives rise to non-transgenic plants that can be commercially sold without a need for regulatory approval.

In the research leading to the present invention, plants of the Cucurbitaceae family were developed with novel mutations in the CENH3 gene that have a haploid inducer effect. It was surprisingly found that these new mutants when crossed to a wild-type plant having 2n chromosomes produce progeny, at least 0.1% of which have n chromosomes.

The present invention thus provides a mutant plant of the Cucurbitaceae family comprising a modified CENH3 gene, which mutant plant when crossed to a wild-type plant having 2n chromosomes produces progeny, at least 0.1% of which have n chromosomes. The mutant plant of the invention can either be used as a female parent or as a male parent in a cross, and in both cases haploid progeny can be obtained.

The invention further relates to parts of the plants, to seeds and to other propagation material, and to progeny of the plants. The parts, seeds, propagation material and progeny comprise the said mutations in their genome.

Suitably, the modified CENH3 gene of the present invention is not naturally occurring, and it comprises a mutation that has been induced by man. Mutations may be introduced into a DNA sequence of a plant genome by a number of methods known in the art. Random mutagenesis comprises the use of chemical compounds to induce mutations (such as ethyl methanesulfonate, nitrosomethylurea, hydroxylamine, proflavine, N-methyl-N-nitrosoguanidine, N-ethyl-N-nitrosourea, N-methyl-N-nitro-nitrosoguanidine, diethyl sulfate, ethylene imine, sodium azide, formaline, urethane, phenol and ethylene oxide), the use of physical means to induce mutations (such as UV-irradiation, fast-neutron exposure, X-rays, gamma irradiation), and the insertion of genetic elements (such as transposons, T-DNA, retroviral elements). Mutations may also be introduced in a targeted, controlled manner, by means of homologous recombination, oligonucleotide-based mutation induction, zinc-finger nucleases (ZFNs), transcription activator-like effector nucleases (TALENs) or Clustered Regularly Interspaced Short Palindromic Repeat (CRISPR) systems (such as CRISPR-Cas9 or CRISPR-Cpf1).

The presence of a mutation in a plant genome may be detected by a number of different techniques known in the prior art, including but not limited to DNA-sequencing, RNA-sequencing, SNP microarray, Restriction Fragment Length Polymorphism (RFLP), Invader® assay, KASP™ assay, TaqMan™ assay.

The term “modified CENH3 gene” refers to a CENH3 gene that is a non-naturally occurring variant of a naturally-occurring (wild-type) CENH3 gene, which comprises at least one non-synonymous nucleotide change relative to a corresponding wild-type CENH3 gene and which encodes a modified CENH3 protein. A non-synonymous nucleotide change is a point mutation in a coding nucleotide sequence that alters the amino acid sequence of the protein for which it codes. This can be either a missense mutation, which is a point mutation in which a single nucleotide change results in a codon that codes for a different amino acid than in the corresponding wild-type sequence, or it can be a non-sense mutation, which is a point mutation in which a single nucleotide change results in the change of a codon to a premature stop codon. A missense mutation leads to the expression of a modified CENH3 protein with at least one amino acid change when compared to the corresponding wild-type protein, and a non-sense mutation leads to the expression of a modified CENH3 protein that is truncated when compared to the corresponding wild-type protein.

The term “modified CENH3 protein” refers to a CENH3 protein that is a non-naturally occurring variant of a naturally-occurring (wild-type) CENH3 protein, which comprises at least one amino acid change or a premature stop codon, when compared to the corresponding wild-type protein sequence.

The modified CENH3 gene of the invention suitably comprises at least one mutation compared to an otherwise identical naturally occurring CENH3 gene, which at least one mutation gives rise to at least one amino acid change in the encoded protein or to the occurrence of a premature stop codon in the encoded modified CENH3 protein.

In a preferred embodiment, the modification in the modified CENH3 protein comprises a mutation in the Histone Fold Domain (FIG. 2), which mutation affects the function of the encoded CENH3 protein. In one embodiment said mutation is a non-sense mutation, i.e. it causes the occurrence of a premature stop-codon (TAA, TAG or TGA), leading to the expression of a shorter, truncated version of the encoded protein. In another embodiment said mutation causes an amino acid change in the encoded protein, such that the normal function of the encoded protein is impaired.

Preferably, the modified CENH3 protein comprises an amino acid change that is predicted to be not tolerated in view of the biological function of the protein. The effect of an amino acid substitution in the context of a given protein can be predicted in silico, e.g. with SIFT (Ng and Henikoff, 2001, Genome Res. 11: 863-874).

A “not tolerated” amino acid change may occur when an amino acid is replaced by another amino acid that has different chemical properties, i.e. a non-conservative amino acid substitution, also termed a non-conservative amino acid change (for example, when a hydrophobic, non-polar amino acid such as Ala, Val, Leu, Ile, Pro, Phe, Trp or Met is replaced by a hydrophilic, polar amino acid, such as Gly, Ser, Thr, Cys, Tyr, Asn or Gln, or when an acidic, negatively charged amino acid such as Asp or Glu is replaced by a basic, positively charged amino acid, such as Lys, Arg or His).

In one embodiment, the plant of the invention is a Cucumis sativus (cucumber) plant having a premature stop codon at position 102 of the CENH3 protein sequence (SEQ ID No:1), resulting from the mutation of a CAA codon (encoding glutamine, Q) at that position in the coding gene sequence into a TAA stop codon. Said plant expresses a truncated version of the CENH3 protein (SEQ ID No:4).

The present invention also relates to a Cucumis melo (melon) plant having a premature stop codon at position 102 of the CENH3 protein sequence (SEQ ID No:2), resulting from the mutation of a CAA codon (encoding glutamine, Q) at that position in the coding gene sequence into a TAA stop codon. Said plant expresses a truncated version of the CENH3 protein (SEQ ID No:5).

The present invention further relates to a Citrullus lanatus (watermelon) plant having a premature stop codon at position 122 of the CENH3 protein sequence (SEQ ID No:3), resulting from the mutation of a CAA codon (encoding glutamine, Q) at that position in the coding gene sequence into a TAA stop codon. Said plant expresses a truncated version of the CENH3 protein (SEQ ID No:6).

The present invention also relates to a plant of the Cucurbitaceae family having a premature stop codon at the position that corresponds to position 102 in the orthologous protein from cucumber or melon and to position 122 in the orthologous protein from watermelon, as shown in the alignment of FIG. 1, suitably resulting from the mutation of a CAA or CAG codon (encoding glutamine, Q) at that position in the coding gene sequence into a TAA or TAG stop codon.

In another embodiment, said plant is a Cucumis sativus (cucumber) plant having Valine at position 115 of the CENH3 protein sequence (SEQ ID No:1), resulting from the mutation of a GAT codon (encoding Aspartate, D) to a GTT codon (encoding Valine, V) at that position in the coding gene sequence. The modified protein sequence is SEQ ID No:7.

The present invention also relates to a Cucumis melo (melon) plant having Valine at position 115 of the CENH3 protein sequence (SEQ ID No:2), resulting from the mutation of a GAC codon (encoding Aspartate, D) to a GTC codon (encoding Valine, V) at that position in the coding gene sequence. The modified protein sequence is SEQ ID No:8.

The present invention further relates to a Citrullus lanatus (watermelon) plant having Valine at position 135 of the CENH3 protein sequence (SEQ ID No:3), resulting from the mutation of a GAT codon (encoding Aspartate, D) to a GTT codon (encoding Valine, V) at that position in the coding gene sequence. The modified protein sequence is SEQ ID No:9. In the CENH3 protein from watermelon, position 135 corresponds to position 115 in the orthologous protein from cucumber and melon, as can be seen in FIG. 1.

The present invention also relates to a plant of the Cucurbitaceae family having Valine at the position that corresponds to position 115 in the orthologous protein from cucumber or melon and to position 135 in the orthologous protein from watermelon, as shown in the alignment of FIG. 1, suitably resulting from the mutation of a CAA or CAG codon (encoding glutamine, Q) at that position in the coding gene sequence into a TAA or TAG stop codon.

The wild-type coding DNA-sequences (CDS) from cucumber, melon and watermelon can be found under SEQ ID No:10, 11 and 12, respectively, and the codons referred to in the text above are underlined therein.

The present invention thus provides a mutant plant of the Cucurbitaceae family comprising a modified CENH3 gene, which mutant plant when crossed to a wild-type plant having 2n chromosomes produces progeny, at least 0.1% of which have n chromosomes, wherein said modification preferably leads to the occurrence of a premature stop codon or to a non-conservative amino acid change in the Histone Fold Domain of the encoded CENH3 protein.

The present invention further provides a mutant cucumber plant comprising a modified CENH3 gene that encodes a protein that corresponds to SEQ ID No:4 or SEQ ID No:7, which mutant cucumber plant when crossed to a wild-type cucumber plant having 2n chromosomes produces progeny, at least 0.1% of which have n chromosomes.

The invention also provides a mutant melon plant comprising a modified CENH3 gene that encodes a protein that corresponds to SEQ ID No:5 or SEQ ID No:8, which mutant melon plant when crossed to a wild-type melon plant having 2n chromosomes produces progeny, at least 0.1% of which have n chromosomes.

The invention further provides a mutant watermelon plant comprising a modified CENH3 gene that encodes a protein that corresponds to SEQ ID No:6 or SEQ ID No:9, which mutant watermelon plant when crossed to a wild-type watermelon plant having 2n chromosomes produces progeny, at least 0.1% of which have n chromosomes.

The present invention also relates to the use of said mutant plants for the production of haploid or doubled haploid plants.

The present invention further relates to a method for the production of haploid or doubled haploid plants, comprising:

a) providing a mutant plant of the Cucurbitaceae family according to the present invention;

b) crossing said mutant plant as one parent with a wild-type plant of the same species as the other parent;

c) growing progeny seeds from the cross;

d) selecting progeny plants with a haploid genome that only comprises chromosomes from the wild-type parent, and progeny plants with a diploid genome that only comprises chromosomes from the wild-type parent;

e) optionally doubling the genome of haploid progeny plants selected in step d).

The present invention also relates to haploid and doubled haploid plants of the Cucurbitaceae family, obtainable by the above-described method.

The present invention also provides a plant belonging to the Cucurbitaceae family harbouring at least one mutation in another centromeric histone protein-encoding gene, in addition to the at least one mutation in the CENH3 gene.

In one embodiment, the at least one mutation in another centromeric histone protein-encoding gene is in the CENP-C (centromere protein C) gene. The present invention thus also provides a mutant plant of the Cucurbitaceae family, comprising a modified CENH3 gene and a modified CENP-C gene, which mutant plant when crossed to a wild-type plant having 2n chromosomes produces progeny, at least 0.1% of which have n chromosomes.

Suitably, the modified CENH3 gene in said mutant plant comprises at least one mutation compared to an otherwise identical naturally occurring CENH3 gene, which at least one mutation gives rise to at least one non-conservative amino acid change in the Histone Fold Domain of the encoded modified CENH3 protein or to the occurrence of a premature stop codon in the encoded modified CENH3 protein. Suitably, the modified CENP-C gene in said mutant plant comprises at least one mutation compared to an otherwise identical naturally occurring CENP-C gene, wherein said mutation leads to the occurrence of a premature stop codon or to a not-tolerated amino acid change, preferably in the C-terminal region of the encoded modified CENP-C protein. The C-terminal region comprises a highly conserved region of about 85 amino acids at the C-terminal end of the CENP-C protein sequence.

The present invention further provides a mutant cucumber plant comprising a modified CENH3 gene that encodes a protein that corresponds to SEQ ID No:4 or SEQ ID No:7 and a modified CENP-C gene that encodes a protein that comprises at least one non-conservative amino acid change or a premature stop codon, preferably in the C-terminal region, when compared to the CENP-C protein of SEQ ID No:13, which mutant cucumber plant when crossed to a wild-type cucumber plant having 2n chromosomes produces progeny, at least 0.1% of which have n chromosomes. The C-terminal region starts at position 646 in the sequence of SEQ ID No:13, and it has been underlined in that sequence.

The present invention also provides a mutant melon plant comprising a modified CENH3 gene that encodes a protein that corresponds to SEQ ID No:5 or SEQ ID No:8 and a modified CENP-C gene that encodes a modified CENP-C protein that comprises at least one not-tolerated amino acid change or a premature stop codon, preferably in the C-terminal region, when compared to the CENP-C protein of SEQ ID No:14, which mutant melon plant when crossed to a wild-type melon plant having 2n chromosomes produces progeny, at least 0.1% of which have n chromosomes. The C-terminal region starts at position 645 in the sequence of SEQ ID No:14, and it has been underlined in that sequence.

The current invention can be applied in plants belonging to the Cucurbitaceae family. This plant family comprises various commercially important genera, such as Cucurbita, Cucumis, Lagenaria, Citrullus, Luffa, Benincasa, Momordica, and Trichosantes. These genera comprise, among others, the following vegetable species: Cucumis spp (cucumber, melon, gherkin), Cucurbita spp (zucchini, pumpkin, squash), Citrullus spp (watermelon), Benincasa cerifera (wax gourd), Lagenaria leucantha (bottle gourd), Luffa acutangula (ridge gourd), Luffa cylindrica (sponge gourd), Momordica charantia (bitter gourd), and Trichosantes cucumerina (snake gourd).

The invention will be further illustrated in the following Examples. In these Examples reference is made to the following figures.

### FIGURES

FIG. 1: alignment of CENH3 protein sequences from melon (Cucumis melo), watermelon (Citrullus lanatus) and cucumber (Cucumis sativus). Stars below the alignment indicate amino acid positions that are identical in the proteins from all three species. Sequence conservation is especially very high in the Histone Fold Domain (which starts with the amino acid motif PGTVAL).

FIG. 2: sequence alignment of the Histone Fold Domain (HFD) region of CENH3 protein sequences from melon (Cucumis melo), watermelon (Citrullus lanatus) and cucumber (Cucumis sativus). The sequence of this domain is almost completely identical in all three species.

### EXAMPLES

### Example 1

### Identification of CENH3 Orthologues in Cucurbitaceae

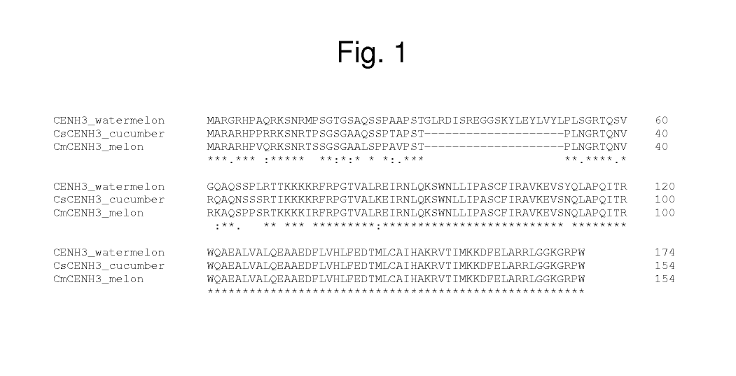

Orthologues of the CENH3 gene were identified in Cucurbitaceae species by using a nucleotide Blasting programme (BLASTN) to compare the conserved histone fold domain of CENH3 with the genomic sequences of crop species of the Cucurbitaceae family. This search resulted in the identification of CENH3 genes and the CENH3 proteins they encode in cucumber (SEQ ID No:1, encoded by SEQ ID No:10), melon (SEQ ID No:2, encoded by SEQ ID No:11) and watermelon (SEQ ID No:3, encoded by SEQ ID No:12). FIG. 1 shows the alignment between these three protein sequences.

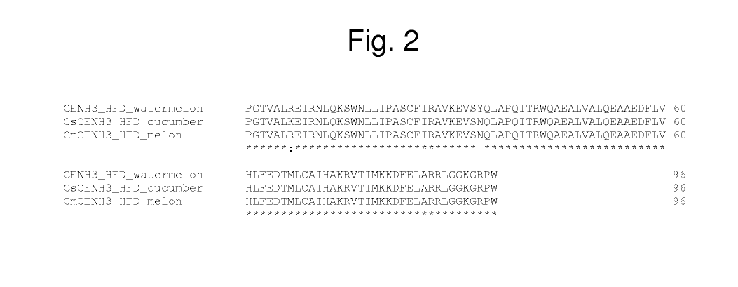

Comparison of the sequences revealed that the HFD region of CENH3 was extremely well conserved in these three commercially important vegetable species belonging to the Cucurbitaceae family. Only for two of the 96 positions in the HFD domain a difference was observed. This is shown in the alignment of FIG. 2. This high degree of conservation indicates that any mutation that is found to cause a haploid-inducer phenotype in one of these species can reliably be expected to cause the same phenotype in the other species. The information obtained from the study of a plant with mutated HFD in CENH3 of one of the Cucurbit species can thus be directly translated to other Cucurbit species, because the effect of that mutation will be identical in the other Cucurbit species.

### Example 2

Identification of a cenh3 Mutant Cucumber Plant with Haploid Inducer Phenotype

Plants of cucumber (Cucumis sativus) line KK 5735 were mutagenised with EMS (ethyl methanesulfonate). In a TILLING approach (Targeting Induced Local Lesions in Genomes), 6144 plants of the EMS-mutagenised population were subsequently screened for point mutations in the CENH3 gene. This screen resulted in the identification of a number of plants with mutations in the HFD of CENH3.

A cucumber plant expressing a mutated CENH3 protein with a premature stop codon at position 102 (where the wild-type sequence has a glutamine, Q, with reference to the amino acid positions in SEQ ID No:1) was identified in this screen, and this plant was found to possess said mutation in a heterozygous state. The CENH3 protein expressed in this mutant plant corresponds to SEQ ID No:4. The mutation was predicted to be functionally not tolerated by SIFT analysis.

This plant was pollinated with pollen from a wild-type cucumber plant, which was genetically distinct from line KK 5735, such that a set of polymorphic molecular markers could be selected with which the two parents of the cross as well as their hybrid progeny could be unambiguously identified by means of molecular marker analysis of their genome.

The fruits resulting from the crosses were harvested, and seeds were collected and sown on agar medium (0.5×MS salts with 10 g L⁻¹ sucrose), and incubated at 25° C. in long-day conditions (16 hours light, 8 hours darkness). When seedlings were big enough, tissue samples were taken from the cotyledons for molecular marker analysis. This analysis revealed that most of the progeny plants were hybrids of mother line KK 5735 and the genetically distinct father line, but about 1% of the progeny plants were shown to be genetically identical to the father line.

These plants were transplanted to soil in the greenhouse for further analysis. Flow cytometry showed that most of these plantlets were haploid, although some of them had spontaneously doubled their genome and had become doubled haploids. The haploid progeny plants were treated with colchicine to induce genome doubling.

### Example 3

Identification of a Cenh3 Mutant Melon Plant with Haploid Inducer Phenotype

Plants of melon (Cucumis melo) Charentais-type line ME 5.176 were mutagenised with EMS (ethyl methanesulfonate). In a TILLING approach (Targeting Induced Local Lesions in Genomes), about 6000 plants of the EMS-mutagenised population were subsequently screened for point mutations in the CENH3 gene. This screen resulted in the identification of a number of plants with mutations in the HFD of CENH3.

A melon plant expressing a mutated CENH3 protein was identified in this screen, in which the amino acid at position 115 of CENH3 was Valine, whereas the wild-type version of this protein in melon (SEQ ID No:2) has Aspartate at that position. The CENH3 protein expressed in this mutant plant corresponds to SEQ ID No:8, and this modified version was termed D115V. The D>V mutation was predicted to be functionally not tolerated by SIFT analysis. This plant was found to be heterozygous for this mutation, and it was selfed to obtain a plant that was homozygous for the mutation.

A melon plant homozygous for the D115V mutation was subsequently pollinated with pollen from a wild-type Charentais melon plant, which was genetically distinct from line ME 5.176, such that a set of polymorphic molecular markers could be selected with which the two parents of the cross as well as their hybrid progeny could be unambiguously identified by means of molecular marker analysis of their genome. The fruits resulting from the crosses were harvested, and seeds were collected and sown on agar medium (0.5×MS salts with 10 g L⁻¹ sucrose), and incubated at 25° C. in long-day conditions (16 hours light, 8 hours darkness). When seedlings were big enough, tissue samples were taken from the cotyledons for molecular marker analysis. This analysis revealed that most of the progeny plants were hybrids of mother line ME 5.176 and the genetically distinct father line, but about 1.5% of the progeny plants were shown to be genetically identical to the father line. These plants were transplanted to soil in the greenhouse for further analysis. Flow cytometry showed that most of these plantlets were haploid, although some of them had spontaneously doubled their genome and had become doubled haploids. The haploid progeny was treated with colchicine to induce genome doubling.

SEQUENCES

>CsCENH3_cucumber
SEQ ID No: 1
MARARHPPRRKSNRTPSGSGAAQSSPTAPSTPLNGRTQNVRQAQNSSS
RTIKKKKRFRPGTVALKEIRNLQKSWNLLIPASCFIRAVKEVSNQLAP
QITRWQAEALVALQEAAEDFLVHLFEDTMLCAIHAKRVTIMKKDFELA
RRLGGKGRPW
>CmCENH3_melon
SEQ ID No: 2
MARARHPVQRKSNRTSSGSGAALSPPAVPSTPLNGRTQNVRKAQSPPS
RTKKKKIRFRPGTVALREIRNLQKSWNLLIPASCFIRAVKEVSNQLAP
QITRWQAEALVALQEAAEDFLVHLFEDTMLCAIHAKRVTIMKKDFELA
RRLGGKGRPW
>CENH3_watermelon
SEQ ID No: 3
MARGRHPAQRKSNRMPSGTGSAQSSPAAPSTGLRDISREGGSKYLEYL
VYLPLSGRTQSVGQAQSSPLRTTKKKKRFRPGTVALREIRNLQKSWNL
LIPASCFIRAVKEVSYQLAPQITRWQAEALVALQEAAEDFLVHLFEDT
MLCAIHAKRVTIMKKDFELARRLGGKGRPW
>CsCENH3_cucumber_Q102*
SEQ ID No: 4
MARARHPPRRKSNRTPSGSGAAQSSPTAPSTPLNGRTQNVRQAQNSSS
RTIKKKKRFRPGTVALKEIRNLQKSWNLLIPASCFIRAVKEVSNQLAP
QITRW
>CmCENH3_melon_Q102*
SEQ ID No: 5
MARARHPVQRKSNRTSSGSGAALSPPAVPSTPLNGRTQNVRKAQSPPS
RTKKKKIRFRPGTVALREIRNLQKSWNLLIPASCFIRAVKEVSNQLAP
QITRW
>CENH3_watermelon_Q122*
SEQ ID No: 6
MARGRHPAQRKSNRMPSGTGSAQSSPAAPSTGLRDISREGGSKYLEYL
VYLPLSGRTQSVGQAQSSPLRTTKKKKRFRPGTVALREIRNLQKSWNL
LIPASCFIRAVKEVSYQLAPQITRW
>CsCENH3_cucumber_D115V
SEQ ID No: 7
MARARHPPRRKSNRTPSGSGAAQSSPTAPSTPLNGRTQNVRQAQNSSS
RTIKKKKRFRPGTVALKEIRNLQKSWNLLIPASCFIRAVKEVSNQLAP
QITRWQAEALVALQEAAEVFLVHLFEDTMLCAIHAKRVTIMKKDFELA
RRLGGKGRPW
>CmCENH3_melon_D115V
SEQ ID No: 8
MARARHPVQRKSNRTSSGSGAALSPPAVPSTPLNGRTQNVRKAQSPPS
RTKKKKIRFRPGTVALREIRNLQKSWNLLIPASCFIRAVKEVSNQLAP
QITRWQAEALVALQEAAEVFLVHLFEDTMLCAIHAKRVTIMKKDFELA
RRLGGKGRPW
>CENH3_watermelon_D135V
SEQ ID No: 9
MARGRHPAQRKSNRMPSGTGSAQSSPAAPSTGLRDISREGGSKYLEYL
VYLPLSGRTQSVGQAQSSPLRTTKKKKRFRPGTVALREIRNLQKSWNL
LIPASCFIRAVKEVSYQLAPQITRWQAEALVALQEAAEVFLVHLFEDT
MLCAIHAKRVTIMKKDFELARRLGGKGRPW
>CsCENH3_cucumber_CDS
SEQ ID No: 10
ATGGCGCGAGCCAGGCATCCACCCCGAAGAAAGTCCAATCGCACGCCA
TCAGGTTCTGGAGCTGCACAGTCTTCCCCAACTGCCCCTTCGACGCCA
CTTAATGGGAGAACGCAAAATGTCAGGCAAGCTCAAAACTCATCGTCA
AGAACAATAAAGAAAAAAAAACGCTTCAGACCAGGGACAGTGGCATTA
AAAGAAATTCGGAATCTCCAGAAATCATGGAATCTGCTAATTCCAGCT
AGCTGTTTCATTCGAGCAGTGAAAGAAGTAAGCAACCAGTTGGCTCCA
CAGATTACGCGTTGGCAAGCTGAAGCTTTAGTAGCTCTTCAGGAAGCA
GCAGAAGATTTTTTGGTTCACCTATTTGAAGATACCATGCTGTGTGCT
ATTCATGCCAAACGTGTAACTATCATGAAAAAGGATTTTGAACTGGCA
CGTCGGTTAGGAGGGAAAGGGAGGCCATGGTGA
>CmCENH3_melon_CDS
SEQ ID No: 11
ATGGCGCGAGCCAGGCATCCAGTCCAAAGAAAGTCCAATCGCACGTCA
TCAGGTTCTGGAGCTGCACTGTCTCCCCCAGCTGTCCCTTCGACGCCA
CTCAATGGGAGAACACAAAATGTCAGGAAAGCTCAAAGCCCACCATCA
AGAACAAAGAAAAAAAAAATACGCTTCAGACCAGGAACGGTGGCATTG
AGAGAAATTCGGAATCTCCAGAAATCATGGAATCTGCTAATTCCAGCT
AGCTGTTTCATCCGAGCAGTGAAAGAAGTAAGCAACCAGTTGGCTCCA
CAGATTACGAGATGGCAAGCTGAAGCTTTAGTAGCTCTTCAGGAAGCC
GCAGAAGACTTTTTGGTTCACCTATTTGAAGATACCATGCTGTGTGCT
ATTCATGCCAAGCGTGTAACTATCATGAAAAAGGATTTTGAACTGGCA
CGTCGATTAGGAGGGAAAGGGAGGCCATGGTGA
>ClCENH3_watermelon_CDS
SEQ ID No: 12
ATGGCGCGAGGGAGGCATCCAGCCCAAAGGAAGTCCAATCGCATGCCA
TCAGGTACTGGATCTGCACAGTCTTCCCCAGCTGCGCCTTCGACGGGC
TTGAGAGATATCTCAAGAGAGGGAGGGTCCAAGTACCTTGAATACTTG
GTTTATCTTCCACTTAGTGGAAGAACACAAAGTGTGGGGCAAGCTCAA
AGCTCACCATTGAGGACAACAAAGAAAAAAAAACGTTTCAGACCAGGG
ACGGTAGCATTGAGGGAAATTCGGAATCTCCAGAAATCATGGAATCTG
CTAATTCCAGCTAGCTGTTTCATCCGAGCAGTGAAAGAAGTAAGCTAC
CAGTTGGCTCCACAGATTACCCGTTGGCAAGCTGAAGCTTTAGTAGCT
CTTCAGGAAGCAGCAGAAGATTTTTTGGTTCATCTATTTGAAGATACC
ATGCTGTGTGCTATTCATGCCAAGCGTGTAACTATCATGAAAAAGGAT
TTTGAACTGGCACGTCGGTTAGGAGGGAAAGGGAGGCCATGGTGA
>CENPC_cucumber
SEQ ID No: 13
MITMANEEARHSDVIDPLAAYSGINLFSTAFGTLPDPSKPHDLGTDLD
GIHKRLKSMVLRSPSKLLEQARSILDGNSNSMISEAATFLVKNEKNEE
ATVKAEENLQERRPALNRKRARFSLKPDARQPPVNLEPTFDIKQLKDP
EEFFLAYEKHENAKKEIQKQTGAVLKDLNQQNPSTNTRQRRPGILGRS
VRYKHQYSSIATEDDQNVDPSQVTFDSGIFSPLKLGTETHPSPHIIDS
EKKTDEDVAFEEEEEEEELVASATKAENRINDILNEFLSGNCEDLEGD
RAINILQERLQIKPLTLEKLCLPDLEAIPTMNLKSSRSNLSKRSLISV
DNQLQKIEILKSKQDNVNLVNPVSTPSSMRSPLASLSALNRRISLSNS
SSDSFSAHGIDQSPSRDPYLFELGNHLSDAVGNTEQSSVSKLKPLLTR
DGGTVANGIKPSKILSGDDSMSNISSSNILNVPQVGGNTALSGTYAST
EAKNVSVSSTDVEINEKLSCLEAQADAVANMQIEDHEGSASEQPKLSE
VDLIKEYPVGIRSQLDQSAATCTENIVDGSSRSSGTEHRDEMEDHEGS
ASEQPKSSKVDVIKEYPVAIQSQLDQSTTTTCAENIADGASRSSGTDH
HDGEQVKPKSRANKQHKGKKISRRQSLAGAGTTWQSGVRRSTRFKTRP
LEYWKGERLLYGRVHESLTTVIGLKYVSPAKGNGKPTMKVKSLVSNEY
KDLVELAALH
>CENPC_melon
SEQ ID No: 14
MTMVNEETRPSDVIDPLAAYSGINLFPTAFGTLTDPSKPHDLGTDLDG
IHKRLKSMVLRSPSKLLEQARSILDGNSKSMISEAATFLVKNEKNEAA
SVKAEENPQERRPALNRKRARFSLKPDAGQPPVNLEPTFDIKQLKDPE
EFFLAYEKHENAKKEIQKQMGAVLKDLNQQNPSTNTRQRRPGILGRSV
RYKHQYSSITTEDDQNVDPSQVTFDSGVFSPLKLGTETHPSPHIIDSE
KKTDEDVAFEEEEEEEELVASATKAENRVNDILDEFLSGNCEDLEGDR
AINILQERLQIKPLTLEKLCLPDLEAIPTMNLKSTRGNLSKRSLISVD
NQLQKTETLKSKEDNENLVNLVSTPSSMRSPLASLSALNRRISLSNSS
GDSFSAHGIDRSPARDPYLFELGNHLSDAVGITEHSSVSKLKPLLTRD
GGTIANGIQPSKILSGDDSMSKISSSNILNVLQVGSNTALSGTYASTD
AKNVSGSSTDVEINEKLSCLEAQADVVANMQIDHQGSASEQPKLSEVD
LIEEYPVGIRSQLDQSAATCTENIVDGSSRSSGTEHHDEMEDHEGSAS
EQPNSSKVDMIKEYPVGIQIQLDQSTTTTTCAEKIVDGTSRSSGTDHH
DEEQVKPKSRANKQRKGKKISGRQSLAGAGTTWKSGVRRSTRFKIRPL
EYWKGERMLYGRVHESLATVIGLKYVSPEKGNGKPTMKVKSLVSNEYK
DLVDLAALH

1. Mutant plant of the Cucurbitaceae family comprising a modified CENH3 gene, which mutant plant, when crossed to a wild-type plant having 2n chromosomes produces progeny, at least 0.1% of which have n chromosomes.

2. Mutant plant as claimed in claim 1, wherein the modified CENH3 gene comprises at least one mutation compared to an otherwise identical naturally occurring CENH3 gene, which at least one mutation gives rise to at least one non-conservative amino acid change in the Histone Fold Domain of the encoded modified CENH3 protein or to the occurrence of a premature stop codon in the encoded modified CENH3 protein.

3. Mutant plant as claimed in claim 1, wherein the plant is a Cucumis sativus plant and the modified CENH3 gene encodes a protein that corresponds to SEQ ID No:4 or SEQ ID No:7.

4. Mutant plant as claimed in claim 1, wherein the plant is a Cucumis melo plant and the modified CENH3 gene encodes a protein that corresponds to SEQ ID No:5 or SEQ ID No:8.

5. Mutant plant as claimed in claim 1, wherein the plant is a Citrullus lanatus plant and the modified CENH3 gene encodes a protein that corresponds to SEQ ID No:6 or SEQ ID No:9.

6. Part of the mutant plant of claim 1, in particular seeds and other propagation material, which part comprises the mutation in its genome.

7. Use of the mutant plant of claim 1 for the production of haploid or doubled haploid plants.

8. Method for the production of haploid or doubled haploid plants, comprising:
a) providing a mutant plant according to claim 1;
b) crossing said mutant plant as one parent with a wild-type plant of the same species as the other parent;
c) growing progeny seeds from the cross;
d) selecting progeny plants with a haploid genome that only comprises chromosomes from the wild-type parent, and progeny plants with a diploid genome that only comprises chromosomes from the wild-type parent;
e) optionally doubling the genome of haploid progeny plants selected in step d).

9. Doubled haploid plants obtainable by the method of claim 8.


SEQUENCE LISTING (17 sequences)

SEQ ID NO: 1 - protein, 154 aa, Cucumis sativus
MARARHPPRRKSNRTPSGSGAAQSSPTAPSTPLNGRTQNVRQAQNSSSRTIKKKKRFRPGTVALKEIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 2 - protein, 154 aa, Cucumis melo
MARARHPVQRKSNRTSSGSGAALSPPAVPSTPLNGRTQNVRKAQSPPSRTKKKKIRFRPGTVALREIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 3 - protein, 174 aa, Citrullus lanatus
MARGRHPAQRKSNRMPSGTGSAQSSPAAPSTGLRDISREGGSKYLEYLVYLPLSGRTQSVGQAQSSPLRTTKKKKRFRPGTVALREIRNLQKSWNLLIPASCFIRAVKEVSYQLAPQITRWQAEALVALQEAAEDFLVHLFEDTMLCAIHAKRVTIMKKDFELARRLGGKGRPW

SEQ ID NO: 4 - protein, 101 aa, Cucumis sativus
MARARHPPRRKSNRTPSGSGAAQSSPTAPSTPLNGRTQNVRQAQNSSSRTIKKKKRFRPGTVALKEIRNLQKSWNLLIPASCFIRAVKEVSNQLAPQITRW

SEQ ID NO: 5 - protein, 101 aa, Cucumis melo
MARARHPVQRKSNRTSSGSGAALSPPAVPSTPLNGRTQNVRKAQSPPSRTKKKKIRFRPGTVALREIRNLQKSWNLLIPASCFIRAVKEVSNQLAPQITRW

SEQ ID NO: 6 - protein, 121 aa, Citrullus lanatus
MARGRHPAQRKSNRMPSGTGSAQSSPAAPSTGLRDISREGGSKYLEYLVYLPLSGRTQSVGQAQSSPLRTTKKKKRFRPGTVALREIRNLQKSWNLLIPASCFIRAVKEVSYQLAPQITRW

SEQ ID NO: 7 - protein, 154 aa, Cucumis sativus
MARARHPPRRKSNRTPSGSGAAQSSPTAPSTPLNGRTQNVRQAQNSSSRTIKKKKRFRPGTVALKEIRNLQKSWNLLIPASCFIRAVKEVSNQLAPQITRWQAEALVALQEAAEVFLVHLFEDTMLCAIHAKRVTIMKKDFELARRLGGKGRPW

SEQ ID NO: 8 - protein, 154 aa, Cucumis melo
MARARHPVQRKSNRTSSGSGAALSPPAVPSTPLNGRTQNVRKAQSPPSRTKKKKIRFRPGTVALREIRNLQKSWNLLIPASCFIRAVKEVSNQLAPQITRWQAEALVALQEAAEVFLVHLFEDTMLCAIHAKRVTIMKKDFELARRLGGKGRPW

SEQ ID NO: 9 - protein, 174 aa, Citrullus lanatus
MARGRHPAQRKSNRMPSGTGSAQSSPAAPSTGLRDISREGGSKYLEYLVYLPLSGRTQSVGQAQSSPLRTTKKKKRFRPGTVALREIRNLQKSWNLLIPASCFIRAVKEVSYQLAPQITRWQAEALVALQEAAEVFLVHLFEDTMLCAIHAKRVTIMKKDFELARRLGGKGRPW

SEQ ID NO: 10 - DNA, 465 bp, Cucumis sativus
  source 1..465: /organism=“Cucumis sativus” /mol_type=“unassigned DNA”
atggcgcgagccaggcatccaccccgaagaaagtccaatcgcacgccatcaggttctggagctgcacagtcttccccaactgccccttcgacgccacttaatgggagaacgcaaaatgtcaggcaagctcaaaactcatcgtcaagaacaataaagaaaaaaaaacgcttcagaccagggacagtggcattaaaagaaattcggaatctccagaaatcatggaatctgctaattccagctagctgtttcattcgagcagtgaaagaagtaagcaaccagttggctccacagattacgcgttggcaagctgaagctttagtagctcttcaggaagcagcagaagattttttggttcacctatttgaagataccatgctgtgtgctattcatgccaaacgtgtaactatcatgaaaaaggattttgaactggcacgtcggttaggagggaaagggaggccatggtga

SEQ ID NO: 11 - DNA, 465 bp, Cucumis melo
  source 1..465: /organism=“Cucumis melo” /mol_type=“unassigned DNA”
atggcgcgagccaggcatccagtccaaagaaagtccaatcgcacgtcatcaggttctggagctgcactgtctcccccagctgtcccttcgacgccactcaatgggagaacacaaaatgtcaggaaagctcaaagcccaccatcaagaacaaagaaaaaaaaaatacgcttcagaccaggaacggtggcattgagagaaattcggaatctccagaaatcatggaatctgctaattccagctagctgtttcatccgagcagtgaaagaagtaagcaaccagttggctccacagattacgagatggcaagctgaagctttagtagctcttcaggaagccgcagaagactttttggttcacctatttgaagataccatgctgtgtgctattcatgccaagcgtgtaactatcatgaaaaaggattttgaactggcacgtcgattaggagggaaagggaggccatggtga

SEQ ID NO: 12 - DNA, 525 bp, Citrullus lanatus
  source 1..525: /organism=“Citrullus lanatus” /mol_type=“unassigned DNA”
atggcgcgagggaggcatccagcccaaaggaagtccaatcgcatgccatcaggtactggatctgcacagtcttccccagctgcgccttcgacgggcttgagagatatctcaagagagggagggtccaagtaccttgaatacttggtttatcttccacttagtggaagaacacaaagtgtggggcaagctcaaagctcaccattgaggacaacaaagaaaaaaaaacgtttcagaccagggacggtagcattgagggaaattcggaatctccagaaatcatggaatctgctaattccagctagctgtttcatccgagcagtgaaagaagtaagctaccagttggctccacagattacccgttggcaagctgaagctttagtagctcttcaggaagcagcagaagattttttggttcatctatttgaagataccatgctgtgtgctattcatgccaagcgtgtaactatcatgaaaaaggattttgaactggcacgtcggttaggagggaaagggaggccatggtga

SEQ ID NO: 13 - protein, 730 aa, Cucumis sativus
MITMANEEARHSDVIDPLAAYSGINLFSTAFGTLPDPSKPHDLGTDLDGIHKRLKSMVLRSPSKLLEQARSILDGNSNSMISEAATFLVKNEKNEEATVKAEENLQERRPALNRKRARFSLKPDARQPPVNLEPTFDIKQLKDPEEFFLAYEKHENAKKEIQKQTGAVLKDLNQQNPSTNTRQRRPGILGRSVRYKHQYSSIATEDDQNVDPSQVTFDSGIFSPLKLGTETHPSPHIIDSEKKTDEDVAFEEEEEEEELVASATKAENRINDILNEFLSGNCEDLEGDRAINILQERLQIKPLTLEKLCLPDLEAIPTMNLKSSRSNLSKRSLISVDNQLQKIEILKSKQDNVNLVNPVSTPSSMRSPLASLSALNRRISLSNSSSDSFSAHGIDQSPSRDPYLFELGNHLSDAVGNTEQSSVSKLKPLLTRDGGTVANGIKPSKILSGDDSMSNISSSNILNVPQVGGNTALSGTYASTEAKNVSVSSTDVEINEKLSCLEAQADAVANMQIEDHEGSASEQPKLSEVDLIKEYPVGIRSQLDQSAATCTENIVDGSSRSSGTEHRDEMEDHEGSASEQPKSSKVDVIKEYPVAIQSQLDQSTTTTCAENIADGASRSSGTDHHDGEQVKPKSRANKQHKGKKISRRQSLAGAGTTWQSGVRRSTRFKTRPLEYWKGERLLYGRVHESLTTVIGLKYVSPAKGNGKPTMKVKSLVSNEYKDLVELAALH

SEQ ID NO: 14 - protein, 729 aa, Cucumis melo
MTMVNEETRPSDVIDPLAAYSGINLFPTAFGTLTDPSKPHDLGTDLDGIHKRLKSMVLRSPSKLLEQARSILDGNSKSMISEAATFLVKNEKNEAASVKAEENPQERRPALNRKRARFSLKPDAGQPPVNLEPTFDIKQLKDPEEFFLAYEKHENAKKEIQKQMGAVLKDLNQQNPSTNTRQRRPGILGRSVRYKHQYSSITTEDDQNVDPSQVTFDSGVFSPLKLGTETHPSPHIIDSEKKTDEDVAFEEEEEEEELVASATKAENRVNDILDEFLSGNCEDLEGDRAINILQERLQIKPLTLEKLCLPDLEAIPTMNLKSTRGNLSKRSLISVDNQLQKTETLKSKEDNENLVNLVSTPSSMRSPLASLSALNRRISLSNSSGDSFSAHGIDRSPARDPYLFELGNHLSDAVGITEHSSVSKLKPLLTRDGGTIANGIQPSKILSGDDSMSKISSSNILNVLQVGSNTALSGTYASTDAKNVSGSSTDVEINEKLSCLEAQADVVANMQIDHQGSASEQPKLSEVDLIEEYPVGIRSQLDQSAATCTENIVDGSSRSSGTEHHDEMEDHEGSASEQPNSSKVDMIKEYPVGIQIQLDQSTTTTTCAEKIVDGTSRSSGTDHHDEEQVKPKSRANKQRKGKKISGRQSLAGAGTTWKSGVRRSTRFKIRPLEYWKGERMLYGRVHESLATVIGLKYVSPEKGNGKPTMKVKSLVSNEYKDLVDLAALH

SEQ ID NO: 15 - protein, 96 aa, Citrullus lanatus
PGTVALREIRNLQKSWNLLIPASCFIRAVKEVSYQLAPQITRWQAEALVALQEAAEDFLVHLFEDTMLCAIHAKRVTIMKKDFELARRLGGKGRPW

SEQ ID NO: 16 - protein, 96 aa, Cucumis sativus
PGTVALKEIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 17 - protein, 96 aa, Cucumis melo
PGTVALREIRNLQKSWNLLIPASCFIRAVKEVSNQLAPQITRWQAEALVALQEAAEDFLVHLFEDTMLCAIHAKRVTIMKKDFELARRLGGKGRPW